(19) United States
(12) Patent Application Publication
(10) Pub. No.: US 2004/0029771 A1
(43) Pub. Date: Feb. 12, 2004

(54) Methods for treating diseases related to intraocular pressure

(75) Inventors: Gregory C. Rigdon, Durham, NC (US); Jonathan W. Stocker, Chapel Hill, NC (US); Grant A. McNaughton-Smith, Morrisville, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Icagen, Inc., Durham, NC

(21) Appl. No.: 10/376,878

(22) Filed: Feb. 28, 2003

Related U.S. Application Data
(60) Provisional application No. 60/403,898, filed on Aug. 15, 2002.
(60) Provisional application No. 60/360,644, filed on Feb. 28, 2002.

(51) Int. Cl. A61K 31/00

(52) U.S. Cl. 514/1

(57) ABSTRACT

This invention relates to the use of compounds able to decrease potassium ion flow through intermediate conductance calcium activated potassium channels for treatment of diseases related to increased intraocular pressure modulated by intermediate conductance calcium activated potassium channels.

### CROSS REFERENCE TO RELATED APPLICATIONS

This application claims priority to U.S. S No. 60/360,644 filed Feb. 28, 2002 and U.S. S No. 60/403,898 filed Aug. 15, 2002, which are both incorporated herein by reference in their entirety.

### FIELD OF THE INVENTION

This invention relates to the use of compounds able to decrease potassium ion flow through intermediate conductance calcium activated potassium channels for treatment of diseases related to increased intraocular pressure modulated by intermediate conductance calcium activated potassium channels.

### BACKGROUND OF THE INVENTION

Ion channels are cellular proteins that regulate the flow of ions, including calcium, potassium, sodium and chloride, into and out of cells. These channels are present in all human cells and affect such physiological processes as nerve transmission, muscle contraction, cellular secretion, regulation of heartbeat, dilation of arteries, release of insulin, and regulation of renal electrolyte transport. Among the ion channels, potassium channels are the most ubiquitous and diverse, being found in a variety of animal cells such as nervous, muscular, glandular, immune, reproductive, and epithelial tissue. These channels allow the flow of potassium in and/or out of the cell under certain conditions. For example, the outward flow of potassium ions upon opening of these channels makes the interior of the cell more negative, counteracting depolarizing voltages applied to the cell. These channels are regulated, e.g., by calcium sensitivity, voltage-gating, second messengers, extracellular ligands, and ATP-sensitivity.

Potassium channels are typically formed by four alpha subunits, and can be homomeric (made of identical alpha subunits) or heteromeric (made of two or more distinct types of alpha subunits). In addition, potassium channels made from Kv, KQT and Slo or BK subunits have often been found to contain additional, structurally distinct auxiliary, or beta, subunits. These subunits do not form potassium channels themselves, but instead they act as auxiliary subunits to modify the functional properties of channels formed by alpha subunits. For example, the Kv beta subunits are cytoplasmic and are known to increase the surface expression of Kv channels and/or modify inactivation kinetics of the channel (Heinemann et al., J. Physiol. 493:625-633 (1996); Shi et al., Neuron 16(4):843-852 (1996)). In another example, the KQT family beta subunit, minK, primarily changes activation kinetics (Sanguinetti et al., Nature 384:80-83 (1996)).

The IK1 channel is a calcium activated channel, also called SK4, KCa4, IKCa, SMIK, and Gardos. The term “IK1” as used herein, refers to both native and cloned intermediate conductance, calcium activated potassium channels. Intermediate conductance, calcium activated potassium channels have been previously described in the literature by their electrophysiology. For example, the Gardos channel, a well known IK channel, is opened by submicromolar concentrations of internal calcium and has a rectifying unit conductance, ranging from 50 pS at −120 mV to 13 pS at 120 mV (symmetrical 120 mM K+; Christopherson, J. Membrane Biol. 119, 75-83 (1991)). IK1 channels are blocked by charybdotoxin (CTX) but not the structurally related peptide iberiotoxin (IBX), both of which block BK channels (Brugnara et al., J. Membr. Biol. 147:71-82 (1995)). IK1 channels are also blocked by maurotoxin. Apamin, a potent blocker of certain native (Vincent et al., J. Biochem. 14:2521 (1975); Blatz & Magleby, Nature 323:718-720 (1986)) and cloned SK channels does not block IK1 channels (de-Allie et al., Br. J. Pharm. 117:479-487 (1996)). The Gardos channel is also blocked by some imidazole compounds, such as clotrimazole, but not ketoconazole (Brugnara et al, 1993, J. Clin. Invest., 92, 520-526). IK1 channels can therefore be distinguished from the other calcium activated potassium channels by their biophysical and pharmacological profiles. IK1 channels from different tissues have been reported to possess a wide range of unit conductance values.

Human IK1 channels have been cloned and characterized (see, e.g., Ishii et al., Proc. Nat'l Acad. Sci. USA 94:11651-11656 (1997); Genbank Accession No. AF0225150; Joiner et al., Proc. Nat'l Acad. Sci. USA 94:11013-11018 (1997); Genbank Accession No. AF000972; Lodsdon et al., J. Biol. Chem. 272:32723-32726 (1997); Genbank Accession No. AF022797; and Jensen et al., Am. J. Physiol. 275:C848-856 (1998); see also WO 98/11139; WO 99/03882; WO 99/25347; and WO 00/12711). Non-human IK1 channels have also been cloned, e.g., from mouse and rat (see, e.g., Vandorpe et al., J. Biol. Chem. 273:21542-21553 (1998); Genbank Accession No. NM_{—}032397; Warth et al., Pflugers Arch. 438:437-444 (1999); Genbank Accession No. AJ133438; and Neylon et al., Circ. Res. (online)85:E33-E43 (1999); Genbank Accession No. AF190458). The gene for the IK1 channel is named KCNN4 and it is located on chromosome 19q13.2 (Ghanshani et al., Genomics 51:160-161 (1998)).

Glaucoma is a disease characterized by increased intraocular pressure. Increased intraocular pressure is associated with many diseases including, but not limited to, primary open-angle glaucoma, normal tension glaucoma, angle-closure glaucoma, acute glaucoma, pigmentary glaucoma, neovascular glaucoma, or trauma related glaucoma., Sturge-Weber syndrome, uveitis, and exfoliation syndrome.

Currently, there are a variety of drugs available that employ different mechanisms to lower intraocular pressure, e.g., timolol, betaxolol, levobunolol, acetazolamide, methazolamide, dichlorphenamide, dorzolamide, brinzolamide, latanoprost, brimonidine, and bimatoprost (see, e.g., U.S. Pat. No. 6,172,054, U.S. Pat. No. 6,172,109, and U.S. Pat. No. 5,652,236). Miotics, beta blockers, alpha-2 agonists, carbonic anhydrase inhibitors, beta adrenergic blockers, prostaglandins and docosanoid are all currently used alone or in combination to treat glaucoma. Miotics and prostaglandins are believed to lower intraocular pressure by increasing drainage of the intraocular fluid, while beta blockers, alpha-2 agonists and carbonic anhydrase are believed to lower intraocular pressure by decreasing production of intraocular fluid thereby reducing the flow of fluid into the eye. All are characterized by side effects ranging from red eye and blurring of vision to decreased blood pressure and breathing difficulties.

In view of the above-described shortcomings in therapy, a need exists for new methods of treating diseases related to increased intraocular pressure. The present invention demonstrates, for the first time, that IK1 channels play an important role in modulating intraocular pressure. Furthermore, the present invention demonstrates that IK1 channel blockers reduce intraocular pressure in animal models. The use of IK1 channels as molecular targets for drugs to treat diseases related to increased intraocular pressure, and the drugs discovered using these methods, are the subject of the present invention.

### SUMMARY OF THE INVENTION

The present invention relates to the use of compounds able to decrease potassium ion flow through IK1 channels for the treatment of diseases related to increased intraocular pressure modulated by potassium channels.

In one aspect, the invention provides a method for reducing intraocular pressure in a subject in need thereof. Intraocular pressure is reduced by decreasing potassium ion flow through IK1 channels in a cell, e.g., a cell capable of mediating the production and/or secretion of aqueous humor. A method for reducing intraocular pressure, therefore, includes treatment methods for subjects in need thereof by administering to a subject a pharmaceutically acceptable carrier and at least one compound able to decrease potassium ion flow through IK1 channels. The composition is administered to the subject in a potassium ion flow decreasing amount.

In one embodiment of the invention, the subject has glaucoma characterized by increased intraocular pressure. In one aspect of the invention, the method prevents glaucoma characterized by increased intraocular pressure. In another aspect of the invention the glaucoma is primary open-angle glaucoma, normal tension glaucoma, angle-closure glaucoma, acute glaucoma, pigmentary glaucoma, neovascular glaucoma, or trauma related glaucoma.

In one embodiment of the invention, the glaucoma is hereditary. In another embodiment, the glaucoma is non-hereditary.

In one aspect of the invention, the subject has increased intraocular pressure associated with Sturge-Weber syndrome. In one embodiment of the invention, the method prevents increased intraocular pressure associated with Sturge-Weber syndrome.

In another aspect of the invention, the subject has increased intraocular pressure associated with uveitis.

In yet another aspect of the invention, the method reduces intraocular pressure to between 12 and 20 mm of mercury. In one embodiment, the method maintains intraocular pressure between 12 and 20 mm of mercury.

In one aspect of the invention, the compound treats chronic elevation of intraocular pressure. In another aspect, it treats acute elevation of intraocular pressure. In yet another aspect of the invention, the compound treats gradual elevation of intraocular pressure.

In another aspect, the invention provides treatment methods for diseases of the eye characterized by increased intraocular pressure.

In one embodiment of the invention, the method prevents destruction of optic nerve cells. In one aspect, the method prevents atrophy of optic nerve cells. In another aspect, the method prevents blindness.

In another embodiment of the invention, the compound treats exfoliation syndrome characterized by increased intraocular pressure. In yet another embodiment, the compound inhibits aqueous humor secretion.

In one aspect of the invention, the subject is a human.

In another aspect of the invention, the IK1 potassium channel is a homomeric channel.

In one embodiment of the invention, the potassium ion flow decreasing amount is 0.001% to 10% w/v. In another embodiment, the potassium flow decreasing amount is 0.1% to 5% w/v. In another embodiment, the potassium ion flow decreasing amount is 10-1000 μg per eye. In another embodiment, the potassium ion flow decreasing amount is 75-150 μg per eye.

In one aspect of the invention, the composition is administered topically.

Another aspect of the invention includes the step of administering to a subject a second or multiple therapeutic agent(s) known to reduce intraocular pressure in a subject. Said agent(s) may be administered with a IK1 inhibitor of the present invention in a single pharmaceutical formulation or as multiple pharmaceutical formulations admixed into a single formulation for ultimate administration to a patient. Suitable intraocular-lowering agents include one or more compounds selected from the group consisting of miotics, sympathomimetics, beta-blockers, alpha-2 agonists, carbonic anhydrase inhibitors, and prostaglandins. Examples of such compounds include timolol, betaxolol, levobunolol, acetazolamide, methazolamide, dichlorphenamide, dorzolamide, brinzolamide, latanoprost, brimonidine, and bimatoprost.

Another aspect of the invention includes the step of administering to the subject a second pharmaceutical composition known to reduce intraocular pressure in a subject. In one embodiment, the second pharmaceutical composition includes as its active ingredient one or more compounds selected from the group consisting of miotics, sympathomimetics, beta-blockers, alpha-2 agonists, carbonic anhydrase inhibitors, and prostaglandins. Examples of such compounds include timolol, betaxolol, levobunolol, acetazolamide, methazolamide, dichlorphenamide, dorzolamide, brinzolamide, latanoprost, brimonidine, and bimatoprost.

In one embodiment of the invention, the compound able to decrease ion flow through IK1 channels has the formula (I):

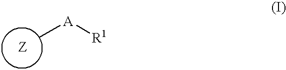

in which the ring system Z is selected from substituted or unsubstituted aryl, and substituted or unsubstituted 5-membered heterocycle. The symbol A represents —NHS(O)₂—, —S(O)₂NH—, —C(R³R⁴)S(O)ₙ—, or —S(O)ₙC(R³R⁴)—, in which R³ and R⁴ are independently selected from hydrogen, substituted or unsubstituted lower alkyl, OR⁵ and —CF₃. The symbol R⁵ represents hydrogen, substituted or unsubstituted lower alkyl, or CF₃. The integer n is selected from 0 to 2. The symbol R¹ represents substituted or unsubstituted aryl, substituted or unsubstituted heteroaryl group, substituted or unsubstituted (C₅-C₇)carbocycle or substituted or unsubstituted (C₅-C₇)heterocycle.

In another embodiment of the invention, the compound able to decrease ion flow through IK1 channels has the formula (I), in which the symbol A represents —NHS(O)₂—.

In another embodiment of the invention, the compound able to decrease ion flow through IK1 channels has the formula (II):

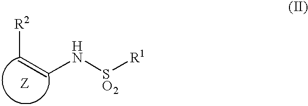

in which the ring system Z is selected from substituted or unsubstituted aryl, and substituted and unsubstituted 5-membered heterocycle. The symbol R¹ represents a substituted or unsubstituted aryl group, a substituted or unsubstituted heteroaryl group, a substituted or unsubstituted (C₅-C₇)carbocycle or a substituted or unsubstituted (C₅-C₇)heterocycle. The symbol R² represents COOR⁶, substituted or unsubstituted 2-furan, substituted or unsubstituted 2-thiazole or

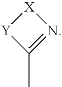

The symbol R⁶ represents a substituted or unsubstituted C₁-C₄ alkyl group, e.g, methyl, ethyl, and —CF₃. X represents —N═N—, —N═C(R⁷)—, —C(R⁷R⁸)—C(R⁷R⁸)— or —C(R⁷)═C(R⁸)—, in which R⁷ and R⁸ independently represent hydrogen, substituted and unsubstituted lower alkyl or —CF3. The symbol Y represents O, NR⁹ or S, in which R⁹ is H, lower alkyl or —CF₃.

In another embodiment of the invention, the compound able to decrease ion flow through IK1 channels is selected from the group consisting of:

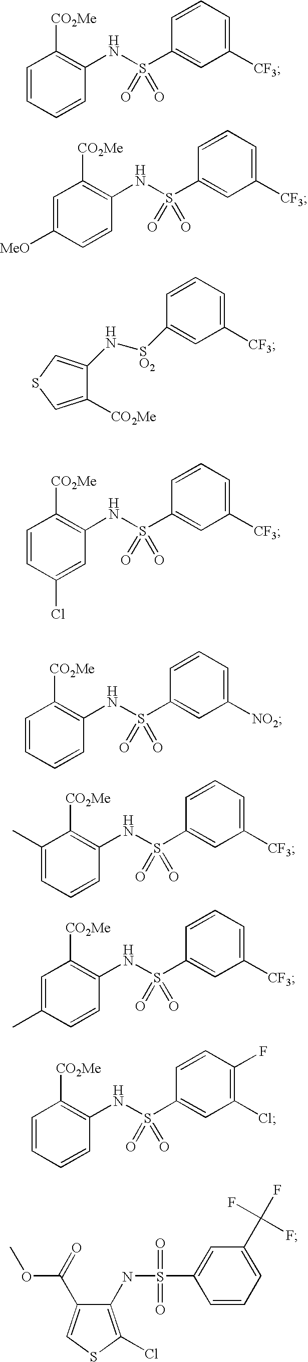

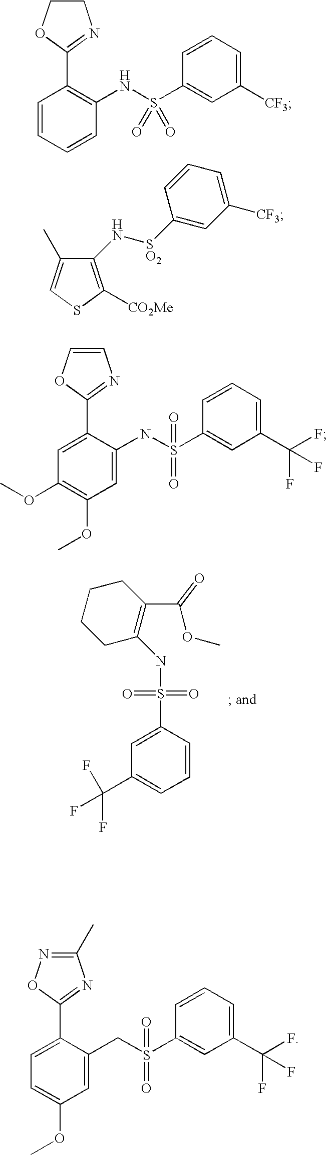

In a preferred embodiment of the invention, the compound able to decrease ion flow through IK1 channels has the formula (III):

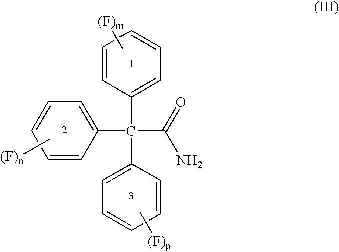

wherein,

m, n and p are independently selected from 0 and 1 and at least one of m, n and p is 1;

when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and

when p is 0, and m is 1 and n is 1, the fluoro substituent at ring 1 is para to the acetamide substituent, and the substituent at ring 2 is located at a position selected from ortho to the acetamide substituent and para to the acetamide substituent.

In another embodiment, the compound able to decrease ion flow through IK1 channels has the formula (IV):

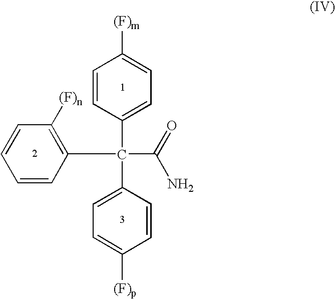

wherein, m, n and p are independently selected from 0 and 1, and at least one of m, n and p is 1.

In another embodiment, the compounds of the invention have a structure according to Formula V:

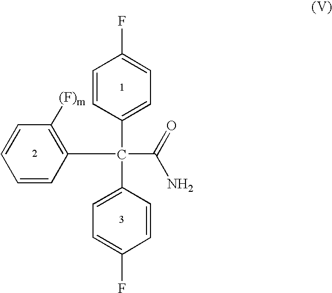

wherein m is either 0 or 1.

In another embodiment of the invention, the compound able to decrease ion flow through IK1 channels is selected from the group consisting of formulas (VI) and (VII):

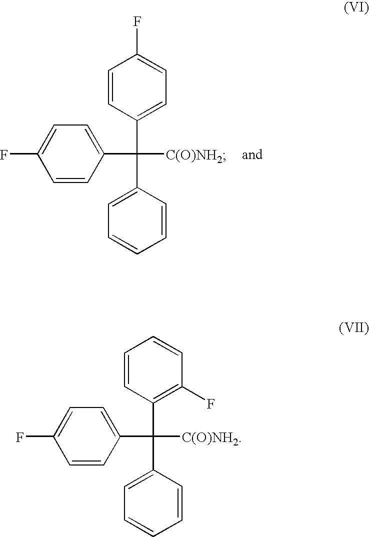

In yet another embodiment of the invention, the compound able to decrease ion flow through IK1 channels has the formula (VIII):

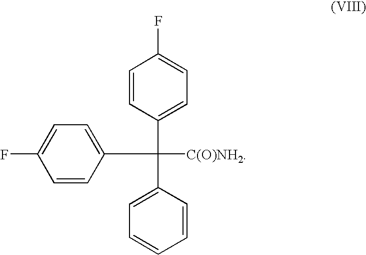

In another aspect, the present invention provides pharmaceutical compositions comprising a pharmaceutically acceptable excipient and a compound of Formula I.

In another aspect, the present invention provides pharmaceutical compositions comprising a pharmaceutically acceptable excipient and a compound of Formula II.

In another aspect, the present invention provides pharmaceutical compositions comprising a pharmaceutically acceptable excipient and a compound of Formula III.

In another aspect, the present invention provides pharmaceutical compositions comprising a pharmaceutically acceptable excipient and a compound of Formula IV.

In another aspect, the present invention provides pharmaceutical compositions comprising a pharmaceutically acceptable excipient and a compound of Formula V.

In another aspect, the present invention provides pharmaceutical compositions comprising a pharmaceutically acceptable excipient and a compound of Formula VI.

In another aspect, the present invention provides pharmaceutical compositions comprising a pharmaceutically acceptable excipient and a compound of Formula VII.

In another aspect, the present invention provides pharmaceutical compositions comprising a pharmaceutically acceptable excipient and a compound of Formula VIII.

In another aspect, the present invention provides pharmaceutical compositions comprising a pharmaceutically acceptable excipient and a compound selected from the group consisting of:

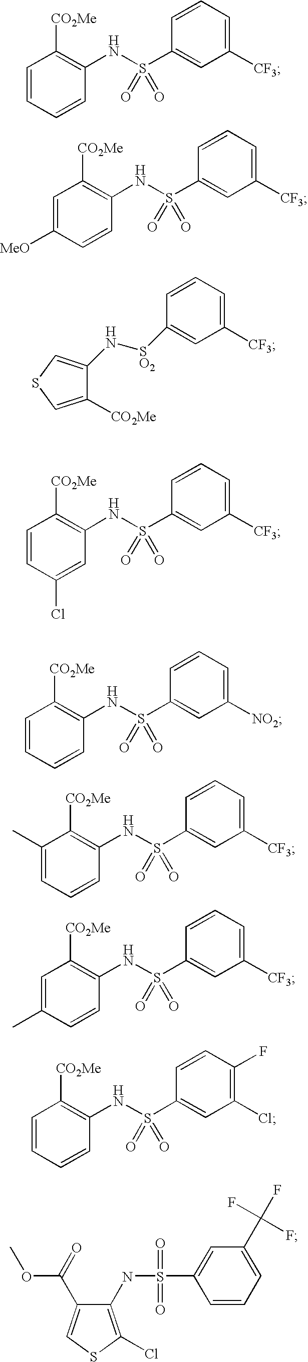

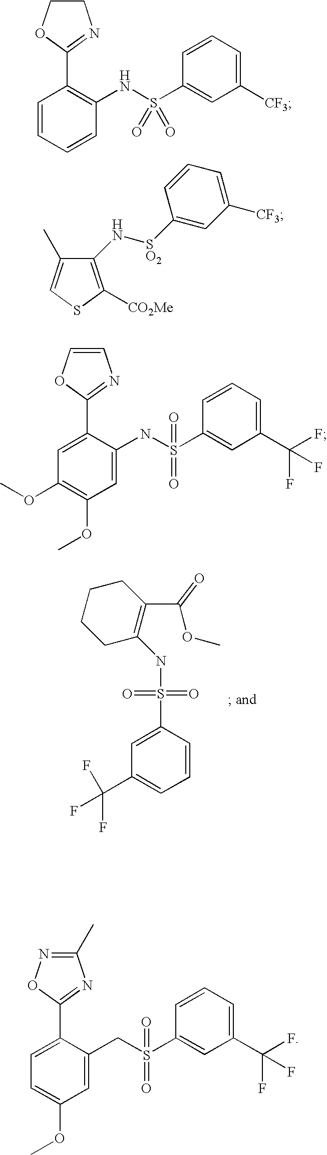

In another aspect of the invention, the compound able to decrease ion flow through IK1 channels has the formula (IX):

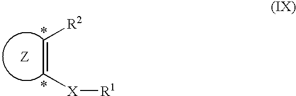

wherein

ring system Z is a member selected from substituted or unsubstituted aryl, unsubstituted carbocycles of from 5 to 7 members, substituted or unsubstituted carbocycles having from 4 to 8 members, substituted or unsubstituted heterocycles having from 4 to 8 members, an d substituted or unsubstituted heteroaryl having from 4 to 8 members;

X is a member selected from the group of-NHS(O)₂—, —S(O)₂NR³—, and —NHC═NR³, wherein R³ is a member selected from H, and substituted or unsubstituted (C₁-C₄)alkyl;

R¹ is a member selected from the group of substituted or unsubstituted aryl, substituted or unsubstituted heteroaryl, substituted or unsubstituted (C₅-C₇)carbocycle and substituted or unsubstituted (C₅-C₇)heterocycle;

R² is a member selected from —Cl, —CF₃, —CO₂R⁴, substituted or unsubstituted aryl, substituted or unsubstituted heterocycle of from 5 to 6 members, and substituted or unsubstituted heteroaryl of from 5 to 6 members, wherein R⁴ is a substituted or unsubstituted (C₁-C₄)alkyl group, which is optionally connected to ring system Z, forming a lactone having from 5 to 7 members; and wherein the double bond between the two carbons marked *, is endocyclic to ring system Z.

In another aspect, the present invention provides pharmaceutical compositions comprising a pharmaceutically acceptable excipient and a compound of Formula IX.

### BRIEF DESCRIPTION OF THE DRAWINGS

FIG. 1 shows the effects of topically administered formula V aqueous suspensions on IOP in normotensive, pigmented rabbits.

### DETAILED DESCRIPTION OF THE INVENTION

Introduction

This application demonstrates for the first time that inhibitors of IK1 channels decrease intraocular pressure. The present invention provides a mechanism for treating diseases related to increased intraocular pressure and provides assays for identifying compounds that inhibit IK1 channels and reduce intraocular pressure. Modulation of IK1 channels therefore represents a novel approach to the treatment of diseases related to increased intraocular pressure. Modulation of IK1 channels can be useful for the treatment of increased intraocular pressure associated with diseases such as glaucoma, Sturge Weber syndrome, exfoliation syndrome, and uveitis. It can also be useful for treating gradual, chronic, and acute elevation of intraocular pressure as well as for preventing the atrophy and destruction of optic nerve cells.

In this invention, compounds able to decrease potassium ion flow through IK1 channels are used to treat increased intraocular pressure. The IK1 channel has been implicated in maintaining ion homeostasis during secretion in a variety of epithelial cells. (Zhang et al., J. Physiol. 499.2:379-389 (1997), Do et al., Invest Ophthalmol Vis. Sci. 41:1853-60 (2000)) However, before the present invention, it was not known that IK1 channels are involved in modulating intraocular pressure.

Aqueous humor, a watery fluid responsible for nourishing the eye and for maintaining intraocular pressure, is secreted by the ciliary epithelium. Current flow across the epithelium regulates the rate of secretion. (Zhang et al., J. Physiol. 499.2:379-389 (1997), Do et al., Invest Ophthalmol Vis. Sci. 41:1853-60 (2000)). The present invention provides, for the first time, methods of treating increased intraocular pressure by administering to subjects compounds able to block IK1 channels. Without being bound by a particular theory, IK1 channels are thought to decrease levels of secretion from the ciliary body. Decreased secretion leads to decreased production of aqueous humor and a corresponding decrease in intraocular pressure. Alternatively, in many patients suffering from diseases related to increased intraocular pressure, the eye is unable to drain the intraocular fluid, creating a buildup of aqueous humor within the anterior chamber of the eye.

In the present invention, compounds have been synthesized that decrease the flow of potassium ions through IK1 channels, e.g., triphenylacetamides and sulfonamides. A preferred compound has the chemical formula V described herein.

To develop pharmaceutically useful IK1 channel inhibitors, candidate compounds must demonstrate acceptable activity towards the target channel. In an initial screen, compounds are judged to be sufficiently potent if they have an IC₅₀ towards the Gardos channel of no less than about 10 micromolar.

In one example, the effect of compounds that decrease potassium ion flow through IK1 channels was tested in vivo in normotensive rabbits (see, e.g., Example 1 herein). Rabbits were administered a suspension of formula V, a triphenylacetamide that blocks IK1 channels, after being maintained on a dark adapted condition on a 12 hour dark/light cycle for 9 to 14 days. Intraocular pressure and pupil diameter measurements were taken. The treated rabbits displayed a significant decrease in intraocular pressure. In this assay, the rabbits showed at least a 2-4 mm decrease in Hg pressure, preferably greater than a 5 mm decrease in Hg pressure.

This assay demonstrates that administration of an IK1 channel blocker reduces intraocular pressure in a subject animal. Thus, IK1 channel inhibitors can be used to treat diseases related to increased intraocular pressure. Such modulators are identified using the in vitro and in vivo assays described herein (see, e.g., WO 00/50026, U.S. Pat. Nos. 6,288,122, 6,028,123, 5,441,957, and 5,273,992; see also Brugnara et al., J. Clin. Invest. 92:520-526 (1993)). In another embodiment, the invention uses an in vitro CHO cell assay, wherein the CHO cells express recombinant IK1, with measurement of radiolabeled rubidium flux as described, e.g., in Brugnara et al., J. Clin. Invest. 92:520-526 (1993). In another embodiment, the compounds of the invention are tested using a in vivo normotensive mammal, e.g., rabbit, assay, described above.

Definitions

As used herein, the following terms have the meanings ascribed to them unless specified otherwise.

The term “intraocular pressure” (IOP) refers to the pressure that is maintained within the eye. The anterior chamber of the eye is bounded by the cornea, iris, pupil and lens. It is filled with aqueous humor, a watery fluid responsible for providing the cornea and lens with oxygen and nutrients. The aqueous humor provides the necessary pressure to help maintain the shape of the eye. When normal secretion of the aqueous humor is interrupted, intraocular pressure is affected.

The phrase “reducing intraocular pressure” refers to reducing the amount of pressure within the eye. The average intraocular pressure in a normal population is 14-16 millimeters of mercury (mmHg). In a normal population, pressures up to 20 mmHg may be in normal range. Pressures of about 22 mm Hg or higher are typically indicative of abnormal intraocular pressure. (Harrison's Principles of Internal Medicine, 1: 168).

The term “intermediate conductance potassium channels” refers to calcium activated potassium channels that are gated by internal calcium ions with a unit conductance of about 20-85 pS (see, e.g., Ishii et al., Proc. Natl. Acad. Sci. 94:1.1651-11656 (1997)). Both native and cloned intermediate conductance potassium channels are useful in the present invention. IK1 potassium channels, IK1 polynucleotides, and IK1 nucleic acids are identified, isolated, expressed, purified, and expressed in recombinant cells according to methods well known to those of skill in the art (see, e.g., WO 98/11139). IK1 potassium channels are heteromeric or homomeric potassium channels composed of at least one alpha subunit from the IK1 polypeptide family, as described below.

“IK1 polypeptide” or “IK1 subunit” (also referred to as SK4, Gardos, KCa4, IKCa, and SMIK) refers to a polypeptide that is a subunit or monomer of a calcium activated, IK potassium channel and a member of the IK/SK gene families. When an IK1 polypeptide is part of an IK1 potassium channel, either a homomeric or heteromeric potassium channel, the channel has intermediate conductance and is gated by internal calcium ions. The term IK1 polypeptide therefore refers to polymorphic variants, alleles, mutants, and interspecies homologs that: (1) have a sequence that has greater than about 60% amino acid sequence identity, preferably about 65, 70, 75, 80, 85, 90, or 95% amino acid sequence identity, to a IK1 gene family member such as those described in, e.g., Ishii et al., Proc. Natl. Acad. Sci. 94:11651-11656 (1997); Joiner et al., Proc. Nat'l Acad. Sci. USA 94:11013-11018 (1997); Logsdon et al., J. Biol. Chem. 272:32723-32726 (1997); WO 98/11139, WO 99/03882; WO 99/25347; and WO 00/12711; (2) bind to antibodies, e.g., polyclonal antibodies, raised against an immunogen comprising a IK1 gene family member polypeptide, as described above, and conservatively modified variants thereof; (3) specifically hybridize under stringent hybridization conditions to a sequence encoding a IK1 gene family member polypeptide, as described above, and conservatively modified variants thereof; or (4) are amplified by primers that specifically hybridize under stringent hybridization conditions a sequence encoding a IK1 gene family polypeptide, as described above.

Exemplary stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. In another example, the stringent conditions include a hybridization in a buffer of 40% formamide, 1 M NaCl, 1% SDS at 37° C., and a wash in 1×SSC at 45° C. For stringent PCR amplification, a temperature of about 62° C. is typical, although stringent annealing temperatures can range from about 50° C. to about 65° C., depending on the primer length and specificity. Typical cycle conditions include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 85%, 90%, or 95% identity), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

Algorithms suitable for determining percent sequence identity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Inhibitors,” “blockers,” or “modulators” of calcium activated potassium channels comprising IK1 refer to inhibitory molecules identified using in vitro and in vivo assays for IK1 channel function. In particular, inhibitors, blockers, and modulators refer to compounds that decrease IK1 channel function, thereby decreasing intraocular pressure in a subject. Inhibitors are compounds that decrease, block, prevent, delay activation, inactivate, desensitize, or down regulate the channel, or speed or enhance deactivation. Such assays for inhibitors and blockers also include, e.g., expressing recombinant IK1 in cells or cell membranes (e.g., CHO cells expressing recombinant IK1 channels) and then measuring flux of ions through the channel directly or indirectly. Alternatively, cells expressing endogenous IK1 channels can be used in such assays.

To examine the extent of inhibition, samples or assays comprising an IK1 channel are treated with a potential inhibitor compound and are compared to control samples without the test compound. Control samples (untreated with test compounds) are assigned a relative IK1 activity value of 100%. Inhibition of channels comprising IK1 is achieved when the IK1 activity value relative to the control is about 90%, preferably 50%, more preferably 25-0%. An amount of compound that activates or inhibits a IK1 channel, as described above, is a “potassium channel modulating amount” of the compound, which thereby reduces intraocular pressure in a subject.

The phrase “modulating ion flow,” or “decreasing ion flow” in the context of assays for compounds affecting ion flux through an IK1 channel, for the purposes of reducing intraocular pressure in a subject, includes the determination of any parameter that is indirectly or directly under the influence of the channel. It includes physical, functional and chemical effects, e.g., changes in ion flux including radioisotopes, current amplitude, membrane potential, current flow, transcription, protein binding, phosphorylation, dephosphorylation, second messenger concentrations (cAMP, cGMP, Ca2+, IP3), ligand binding, and other physiological effects such as changes in voltage and current. The ion flux can be any ion that passes through a channel and analogues thereof, e.g., potassium, rubidium, sodium. Such functional, chemical or physical effects can be measured by any means known to those skilled in the art, e.g., patch clamping, voltage-sensitive dyes, whole cell currents, radioisotope efflux, inducible markers.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

For the compounds able to decrease ion flow through IK1 channels, the term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e., C1-C10 means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups which are limited to hydrocarbon groups are termed “homoalkyl”.

The term “alkylene” by itself or as part of another substituent means a divalent radical derived from an alkane, as exemplified by —CH2CH2CH2CH2—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH2-CH2-O—CH3, —CH2-CH2-NH—CH3, —CH2-CH2-N(CH3)—CH3, —CH2-S—CH2-CH3, —CH2-CH2, —S(O)—CH3, —CH2-CH2-S(O)2—CH3, —CH═CH—O—CH3, —Si(CH3)3, —CH2-CH═N—OCH3, and —CH═CH—N(CH3)—CH3. Up to two heteroatoms may be consecutive, such as, for example, —CH2-NH—OCH3 and —CH2-O—Si(CH3)3. Similarly, the term “heteroalkylene” by itself or as part of another substituent means a divalent radical derived from heteroalkyl, as exemplified by —CH2-CH2-S—CH2CH2- and —CH2-S—CH2-CH2-NH—CH2-. For heteroalkylene groups, heteroatoms can also occupy either or both of the chain tennini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C1-C4)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″ R′″, —OC(O)R′, —C(O)R′, —CO2R′, —CONR′R″, —OC(O)NR′R″, —NR″C(O)R′, —NR′—C(O)NR″R′″, —NR″C(O)2R′, —NH—C(NH2)═NH, —NR′C(NH2)═NH, —NH—C(NH2)═NR′, —S(O)R′, —S(O)2R′, —S(O)2NR′R″, —CN and —NO2 in a number ranging from zero to (2m′+1), where m′ is the total number of carbon atoms in such radical. R′, R″ and R′″ each independently refer to hydrogen, unsubstituted (C1-C8)alkyl and heteroalkyl, unsubstituted aryl, aryl substituted with 1-3 halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF3 and —CH2CF3) and acyl (e.g., —C(O)CH3, —C(O)CF3, —C(O)CH2OCH3, and the like).

Similarly, substituents for the aryl and heteroaryl groups are varied and are selected from: -halogen, —OR′, —OC(O)R′, —NR′R″, —SR′, —R′, —CN, —NO2, —CO2R′, —CONR′R″, —C(O)R′, —OC(O)NR′R″, —NR″C(O)R′, —NR″C(O)2R′, —NR′—C(O)NR″R′″, —NH—C(NH2)═NH, —NR′C(NH2)═NH, —NH—C(NH2)═NR′, —S(O)R′, —S(O)2R′, —S(O)2NR′R″, —N3, —CH(Ph)2, perfluoro(C1-C4)alkoxy, and perfluoro(C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

Two of the substituents on adjacent atoms of the aryl or heteroaryl ring may optionally be replaced with a substituent of the formula -T-C(O)—(CH2)q-U—, wherein T and U are independently —NH—, —O—, —CH2- or a single bond, and q is an integer of from 0 to 2. Alternatively, two of the substituents on adjacent atoms of the aryl or heteroaryl ring may optionally be replaced with a substituent of the formula -A-(CH2)x-B—, wherein A and B are independently —CH2-, —O—, —NH—, —S—, —S(O)—, —S(O)2—, —S(O)2NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH2)s-X—(CH2)t-, where s and t are independently integers of from 0 to 3, and X is —O—, —NR′—, —S—, —S(O)—, —S(O)2—, or —S(O)2NR′. The substituent R′ in —NR′— and —S(O)2NR′— is selected from hydrogen or unsubstituted (C1-C6)alkyl.

As used herein, the term “heteroatom” is meant to include oxygen (O), nitrogen (N), sulfur (S) and silicon (Si).

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (3H), iodine-125 (125I) or carbon-14 (14C). All isotopic variations of the compounds of the present invention, whether radioactive or not, are intended to be encompassed within the scope of the present invention.

The term “glaucoma” refers to an optic neuropathy or degenerative state usually associated with elevation of intraocular pressure. See, Shields, TEXTBOOK OF GLAUCOMA (4th Ed.), 1997, Lippincott, Williams and Wilkins, which is incorporated herein by reference. The mechanism by which elevated eye pressure injures the optic nerve is not well understood. It is known that axons entering the inferotemporal and superotemporal aspects of the optic disc are damaged. As fibers of the disc are destroyed, the neural rim of the optic disc shrinks and the physiologic cup within the optic disc enlarges. A term known as pathologic “cupping” refers to this shrinking and enlarging process. Although it is possible to measure the cup-to-disc ratio, it is not a useful diagnostic tool because it varies widely in the population. However, it can be used to measure the progression of the disease by a series of measurements in a time period.

Glaucoma is not a single disease but a group of conditions with various causes. In most cases, these conditions produce increased pressure within the eye. Ultimately glaucoma can lead to optic nerve damage and the loss of visual function. It is not unusual for persons who exhibit gradual development of intraocular pressure to exhibit no symptoms until the end-stage of the disease is reached.

The term “open angle glaucoma”—refers to a chronic type of glaucoma. Occurring in approximately 1% of Americans, open-angle glaucoma is the most common type of glaucoma. Open-angle glaucoma is characterized by a very gradual, painless rise of pressure within the eye. Subjects with open-angle glaucoma exhibit no outward manifestations of disease until irreversible vision impairment.

“Normal tension glaucoma” commonly referred to as low tension glaucoma is a form of open angle glaucoma that accounts for about ⅓ of open-angle glaucoma cases in the United States.

“Angle closure glaucoma” is a glaucoma most prevalent in people who are far-sighted. In angle closure glaucoma, the anterior chamber of the eye is smaller than average hampering the ability of the aqueous humor to pass between the iris and the lens on its way to the anterior chamber, causing fluid pressure to build up between the iris.

“Acute glaucoma” is caused by a sudden increase in intraocular pressure. This intense rise in pressure is accompanied by severe pain. In acute glaucoma, there are outward manifestations of the disease including red eye, cornea swelling and clouding over.

The term “pigmentary glaucoma” refers to a hereditary condition which develops more frequently in men than in woman and begins in the twenties or thirties, pigmentary glaucoma affects persons of near-sightedness. Myopic eyes have a concave-shaped iris creating an unusually wide angle. The wideness of the angle causes the pigment layer of the eye to rub on the lens when the pupil constricts and dilates during normal focusing. The rubbing action ruptures the cells of the iris pigment epithelium, thereby releasing pigment particles into the aqueous humor and trabecular meshwork. If pigment plugs the pores of the trabecular meshwork, drainage is inhibited.

The term “exfoliation syndrome” refers to a type of glaucoma most common in persons of European descent. Exfoliation syndrome is characterized by a whitish material that builds on the lens of the eye. Movement of the iris causes this material to be rubbed off the lens along with some pigment from the iris. Both the pigment and the whitish exfoliation material clog the meshwork, inhibiting drainage of the aqueous humor.

The term “trauma related glaucoma” refers to a type of glaucoma caused by an external force acting upon the eye, i.e., chemical burn, blow to the eye. Trauma related glaucoma occurs when this external force causes a mechanical disruption or physical change with in the eye's drainage system.

“Congenital glaucoma” occurs in about 1 in 10,000 births. It may appear up until age 4. Primary congenital glaucoma is due to abnormal development of the trabecular meshwork. Congenital glaucoma can be hereditary as well as non-hereditary. In congenital glaucoma, the eye enlarges or the cornea becomes hazy. The stretching of the cornea causes breaks to occur in the inner lining. The breaks allow aqueous humor to enter the cornea causing it to swell. As the cornea continues to stretch, more aqueous humor is let in and there is an increase in edema and haze in the cornea.

The term “Sturge-Weber Syndrome” refers to a rare syndrome characterized by a facial birthmark which is port wine in color. The birthmark is associated with an abnormal blood vessels on the surface of the brain. These vascular malformations may affect the eyelids, sclera, conjunctiva, and iris. One third of patients with Sturge-Weber syndrome suffer from increased intraocular pressure. This increased pressure leads to glaucoma. A vascular malformation of the sclera causes elevated pressure in the veins. This elevated pressure in the veins drains the eye thereby causing the intraocular pressure to rise and resulting in damage to the drainage system of the eye.

The term “uveitis” refers to a disease characterized by inflammation of the choroid, ciliary body and iris. In anterior uveitis, a decrease in aqueous humor formation may cause dangerously low levels of pressure within the eye. In other forms of uveitis, i.e., posterior uveitis, the intraocular pressure is elevated. The elevation may be caused by active inflammation, insufficient antiinflammatory therapy, excessive corticosteroid use or insufficient glaucoma therapy. If the inflammation is chronic and not properly controlled, it can lead to trabecular cell death.

The term “chronic elevation” refers to increased pressure caused by a condition that is reoccurring and not treatable.

The term “acute elevation” refers to a sudden increase in intraocular eye pressure. The sudden rise can occur within hours and causes pain within the eye and may even cause nausea and vomiting

The term “gradual elevation” refers to a slow increase of pressure within the eye. There are no symptoms associated with the increased rise.

An “ophthalmically acceptable carrier” is a carrier that has substantially no long term or permanent detrimental effect on the eye to which it is administered.

Assays for Compounds that Decrease Ion Flow through IK1 Channels

To develop pharmaceutically useful intermediate conductance, calcium activated potassium channel inhibitors, candidate compounds must demonstrate acceptable activity towards the target channel. The activity of the compounds of the invention towards these ion channels, such as the Gardos channel, can be assayed utilizing methods known in the art.

The activity of a potassium channel comprising an intermediate conductance, calcium activated potassium channel can be assessed using a variety of in vitro and in vivo assays. In one embodiment, the in vivo assays conducted in mammals and disclosed herein, e.g., the rabbit assay in the examples section, are used to identify IK1 channel blockers for treatment of increased intraocular pressure. In another embodiment, the in vitro assays described herein are used, e.g., radiolabeled rubidium flux. Such assays are used to test for inhibitors of IK1 channels and for the identification of compounds that reduce intraocular pressure in a subject. Assays for modulatory compounds include, e.g., measuring current; measuring membrane potential; measure ion flux; e.g., potassium or rubidium; measuring potassium concentration; measuring second messengers and transcription levels; using potassium-dependent yeast growth assays; measuring intraocular pressure, e.g., by administering a compound able to decrease ion flow through IK1 channels to a subject and measuring changes in intraocular pressure.

Other methods for assaying the activity of ion channels and the activity of agents that affect the ion channels are known in the art. The selection of an appropriate assay method is well within the capabilities of those of skill in the art. See, for example, Hille, IONIC CHANNELS OF EXCITABLE MEMBRANES, Sinaner Associates, Inc. Sunderland, Mass. (1992).

Compounds that decrease ion flow through intermediate conductance calcium activated potassium channels are tested using biologically active IK1 channels, either recombinant or naturally occurring. Intermediate conductance calcium activated potassium channels, preferably human IK1 channels can be found in native cells, isolated in vitro, co-expressed or expressed in a cell, or expressed in membrane derived from a cell. Modulation by a compound is tested using standard in vitro or in vivo assays such as those well known in the art or as otherwise described herein. Compounds that decrease the flux of ions will cause a detectable decrease in the ion current density by decreasing the probability of the IK1 channel being open, by increasing the probability of it being closed, by decreasing conductance through the channel, and by hampering the passage of ions.

Decreased flux of potassium may be assessed by determining changes in polarization (i.e., electrical potential) of a cell which expresses, for example, the intermediate conductance, calcium activated potassium ion channel known as the Gardos channel. One method of determining changes in cellular polarization is the voltage-clamp technique e.g., the “cell attached” mode, the “inside out” mode, and the “whole cell” mode (see, e.g., Ackerman et al., New Engl. J. Med. 336:1575-1595 (1997)). Other known assays include radiolabeled rubidium flux assays and fluorescence assays using voltage-sensitive dyes. See, e.g., Vestergarrd-Bogind et al., J. Membrane Biol., 88:67-75 (1988); Danel et al., J. Pharmacol. Meth., 25:185-193 (1991); Holevinsky et al., J. Membrane Biology, 137:59-70 (1994). Assays for compounds capable of inhibiting or increasing potassium flux through the IK1 channel protein can be performed by application of the compounds to a bath solution in contact with and comprising cells having said channel. See, e.g., Blatz et al., Nature, 323:718-720 (1986); Park, J. Physiol., 481:555-570 (1994). Generally the compounds to be tested are present in the range from 1 pM to 100 mM. Changes in function of the channels can be measured in the electrical currents or ionic flux, or by the consequences of changes in currents and flux.

The effects of the test compounds upon the function of the channels can be measured by changes in the electrical currents or ionic flux or by the consequences of changes in currents and flux. Changes in electrical current or ion flux are measured either by increases or decreases in flux of cations such as potassium or rubidium ions. The cations can be measured in a variety of standard ways. They can be measured directly by concentration changes of the ions or indirectly by membrane potential or by radiolabeling of the ions. Consequences of the test compound on ion flux can be quite varied. Accordingly, any suitable physiological parameter can be used to assess the influence of a test compound on the channels of this invention. Changes in channel function can be measured by ligand displacement such as CTX release. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immune-responses (e.g., T cell activation), changes in cell metabolism such as cell growth or pH changes.

Modulators of IK1 Channels

The compounds of the invention, which decrease ion flux through IK1 potassium channels, identified according to the in vitro and in vivo assays described herein and with methodology well known to those of skill in the art. The compounds of the invention are made according to methodology well known to those of skill in the art and as described below. For example, the triphenylacetamide compounds of the invention can be synthesized as described in U.S. Pat. No. 6,288,122.

The compounds tested as modulators of IK1 channels can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, where the cell or tissue expressing an IK1 channel is attached to a solid phase substrate. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds.

Preparation of Potassium Channel Blockers

Compounds of the present invention can be prepared using readily available starting materials or known intermediates. For example, furan derivatized bis-aryl sulfonamides are readily prepared the method of Scheme A:

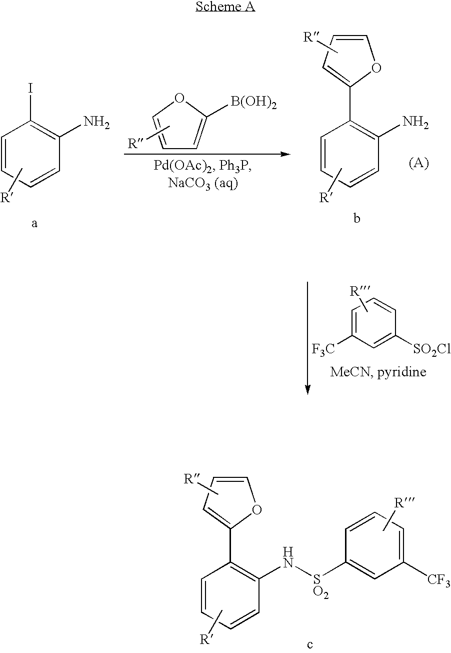

In Scheme A, and each of the succeeding schemes, each of the reaction components can bear one or more substituents (“R groups”) other than a locus of reaction. The symbols R′, R″, R′″, etc. generally represent substituents for aryl or heteroaryl groups as described in the definitions section herein.

In scheme A, the iodo aniline substrate a is coupled with the furan moiety via a Pd mediated reaction with a boronic acid derivative to afford compound b. The resulting adduct is reacted with an activated sulfonic acid derivative to produce adduct c.

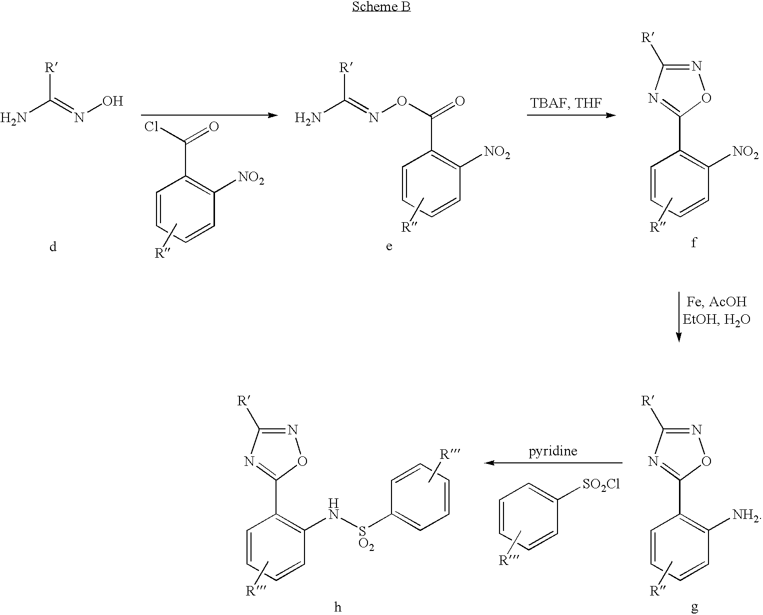

Scheme B sets out an exemplary route to oxadiazolyl-containing compounds of the invention. Thus, amidine d is acylated with a benzoyl chloride species, affording compound e. Compound e is cyclized to compound f. The nitro group of compound f is reduced and the resulting amine is converted to the correspond sulfonamide h.

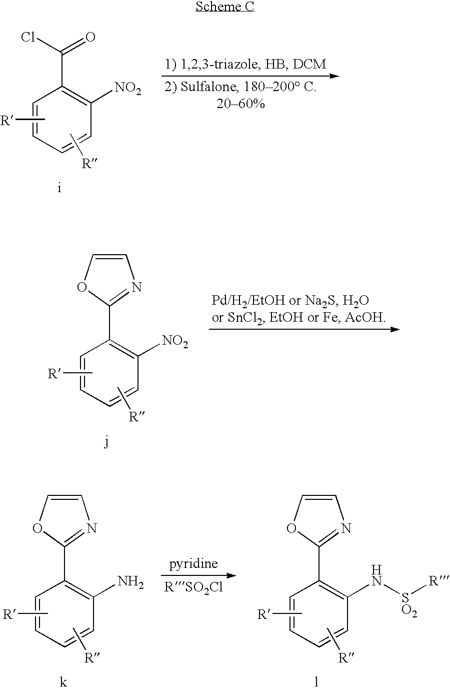

Scheme C sets forth a representative route to oxazole-containing compounds of the invention. Acyl halide i is converted to oxazole j by the action of triazole and sulfalone. The nitro group of j is reduced, affording the corresponding amine k, which is converted to a sulfonamide l by the action of an activated sulfonic acid derivative.

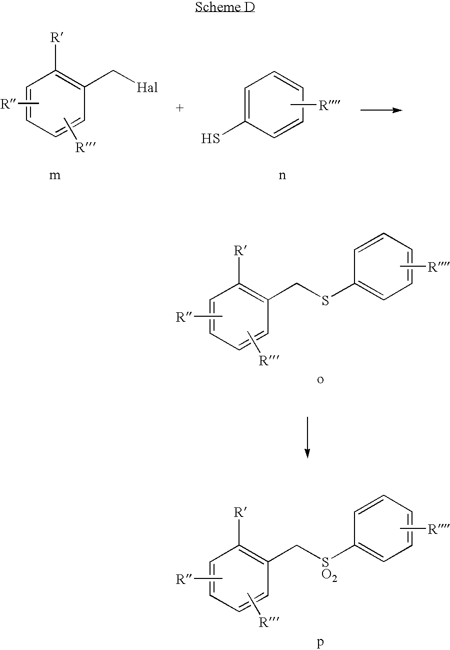

Scheme D provides an exemplary route to bis-aryl sulfones of the invention. Benzyl halide m is reacted with an appropriate thiol n, forming sulfide o, which is subsequently oxidized to sulfone p.

Methods for preparing dimers, trimers and higher homologs of small organic molecules, such as those of the present invention, as well as methods of functionalizing a polyfunctional framework molecule are well known to those of skill in the art. For example, an aromatic amine of the invention is converted to the corresponding isothiocyanate by the action of thiophosgene. The resulting isothiocyanate is coupled to an amine of the invention, thereby forming either a homo- or heterodimeric species. Alternatively, the isothiocyanate is coupled with an amine-containing backbone, such as polylysine, thereby forming a conjugate between a polyvalent framework and a compound of the invention. If it is desired to prepare a hetereofuntionalized polyvalent species, the polylysine is underlabeled with the first isothiocyanate and subsequently labeled with one or more different isothiocyanates. Alternatively, a mixture of isothiocyanates is added to the backbone. Purification proceeds by, for example, size exclusion chromatography, dialysis, nanofiltration and the like.

Pharmaceutical Compositions and Administration

In another aspect, the present invention provides pharmaceutical compositions comprising a pharmaceutically acceptable excipient and a compound of the invention. In one embodiment, the present invention provides pharmaceutical compositions comprising a pharmaceutically acceptable excipient and a compound of Formula II. In another embodiment, the present invention provides pharmaceutical compositions comprising a pharmaceutically acceptable excipient and a compound of Formula III.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical composition. In one embodiment, topical or oral administration and compositions are preferred. In another embodiment, topical administration and compositions are preferred.

Any method of administering drugs directly to a mammalian eye may be employed to administer, in accordance with the present invention, the compound or compounds to the eye to be treated. The primary effect on the mammal resulting from the direct administration of the compound or compounds to the mammal's eye is a reduction in intraocular pressure. More preferably, one or more IK1 blockers and/or additional compounds known to reduce intraocular pressure are applied topically to the eye or are injected directly into the eye. Particularly useful results are obtained when the compound or compounds are applied topically to the eye in an ophthalmic preparation, e.g., as ocular solutions, suspensions, gels or creams, as examples of topical ophthalmic preparations used for dose delivery.

In accordance with the invention the compounds are typically administered in an ophthalmically acceptable carrier in sufficient concentration so as to deliver an effective amount of the compound or compounds to the eye. The compounds are administered in accordance with the present invention to the eye, typically admixed with an ophthalmically acceptable carrier, and optionally with another compound suitable for treatment of glaucoma and/or reduction of intraocular pressure. Any suitable, e.g., conventional, ophthalmically acceptable carrier may be employed including water (distilled or deionized water), saline, and other aqueous media, with or without solubility enhancers such as any of the ophthalmically acceptable beta-cyclodextrins. The compounds may be soluble in the carrier which is employed for their administration, so that the compounds are administered to the eye in the form of a solution. Alternatively, a suspension of the compound or compounds (or salts thereof in a suitable carrier may also be employed.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v. In another embodiment, the dosage range is 10-1000 μg per eye. In another embodiment, the dosage range is 75-150 μg per eye.

When forming compositions for topical administration, the compounds are generally formulated as between about 0.001% to 10% w/v, more preferably between about 0.1% to 5% w/v. In one embodiment, the formulation is 1.0% w/v. In one embodiment, the formulations are solutions in water at a pH preferably between about 7.0 to 7.6 pH, preferably pH 7.4±0.3. In another aspect of the invention, the compounds are formulated as suspensions. In a preferred embodiment, the formulation is in a 1% w/v ophthalmic suspension: 1.0% compound of formula V, micronized; 0.06% carbomer (carbopol 1382), NF; 1.0% poloxamer 188, NF; 2.5% glycerin, USP; 0.01% benzalkonium chloride, NF; sodium hydroxide, NF, q.s. pH 7.4±0.3; and purified water, USP (the formulation may be prepared as % w/w for convenience, and higher grades of water, USP, may be substituted). Other suitable IK1 inhibiting compounds of the invention may be substituted for formula V in this formulation. This formulation may contain additional compounds know to reduce intraocular pressure, or may be administered with additional pharmaceutical compositions.

Various preservatives may be used in an ophthalmic preparation. Preservatives include, but are not limited to, benzalkonium chloride, chlorobutanol, thimerosal, phenylmercuric acetate, and phenylmercuric nitrate. Likewise, various vehicles may be used in such ophthalmic preparation. These vehicles include, but are not limited to, polyvinyl alcohol, povidone, cyclodextrines, hydroxypropyl methyl cellulose, poloxamers, carboxymethyl cellulose and hydroxyethyl cellulose. Such preservatives, if utilized, will typically be employed in an amount between about 0.001 and about 1.0% by weight.

Tonicity adjusters may be added as needed or convenient. They include, but are not limited to, salts, particularly sodium chloride, potassium chloride etc., mannitol and glycerin, or any other suitable ophthalmically acceptable tonicity adjuster. Such agents, if utilized, will typically be employed in an amount between about 0.1 and about 1.0% by weight.

Various buffers and means for adjusting pH may be used so long as the resulting preparation is ophthalmically acceptable. Accordingly, buffers include but are not limited to, acetate buffers, titrate buffers, phosphate buffers, and borate buffers. Acids or bases may be used to adjust the pH of these formulations as needed.

In a similar vein, ophthalmically acceptable antioxidants include, but are not limited to, sodium metabisulfite, sodium thiosulfate, acetylcysteine, butylated hydroxyanisole, and butylated hydroxytoluene.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to increase ocular absorption of the compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The ophthalmic solution (ocular drops) may be administered to the mammalian eye as often as necessary to maintain an acceptable level of intraocular pressure in the eye. In other words, the ophthalmic solution (or other formulation) is administered to the mammalian eye as often as necessary to maintain the beneficial effect of the active ingredient in the eye. Those skilled in the art will recognize that the frequency of administration depends on the precise nature of the active ingredient and its concentration in the ophthalmic formulation. Within these guidelines it is contemplated that the ophthalmic formulation of the present invention will be administered to the mammalian eye once daily. The formulations may be administered to the mammalian eye anywhere from about 1-4× daily, or as otherwise deemed appropriate by the attending physician. The formulations may also be administered in combination with one or more other pharmaceutical compositions known to reduce intraocular pressure in a subject or otherwise have a beneficial effect in a subject, including miotics (e.g., pilocarpine, carbachol, and acetylcholinesterase inhibitors); sympathomimetics (e.g., epinephrine and dipivalylepinephrine); beta-blockers (e.g., betaxolol, levobunolol and timolol); alpha-2 agonists (e.g., para-amino clonidine); carbonic anhydrase inhibitors (e.g., acetazolamide, methazolamide and ethoxzolamide); and prostaglandins and their analogs and derivatives (e.g., latanaprost).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference.

In addition to the above-described principal ingredients, one skilled in formulating ophthalmic compositions will appreciate that ocular compositions may further comprise various pharmaceutically acceptable ingredients, such as antimicrobial preservatives and tonicity agents. Examples of suitable antimicrobial preservatives include: benzalkonium chloride, thimerosal, chlorobutanol, methyl paraben, propyl paraben, phenylethyl alcohol, edetate disodium, sorbic acid, Onamer M.RTM. and other agents equally well-known to those skilled in the art. Such preservatives, if utilized, will typically be employed in an amount between about 0.001 and about 1.0 wt %. Examples of suitable agents which may be used to adjust the tonicity or osmolality of the formulations include: sodium chloride, potassium chloride, mannitol, dextrose, glycerin, and propylene glycol. Such agents, if utilized, will typically be employed in an amount between about 0.1 and about 10.0 wt %. Determination of acceptable amounts of the above adjuvants is readily ascertained by one skilled in the art.

As will likewise be appreciated by those skilled in the art, the compositions may be formulated in various dosage forms suitable for topical ophthalmic delivery, as described above, including solutions, suspensions, emulsions, gels, and erodible solid ocular inserts. The compositions are preferably aqueous suspensions or solutions. Further, such formulated compositions may also include one or more additional active ingredients in a single vial for delivery to the patient. That is to say, in addition to one or more potassium channel inhibitors present in a single formulation, the present invention additionally contemplates the presence of one or more of the following therewith: miotics (e.g., pilocarpine, carbachol, and acetylcholinesterase inhibitors); sympathomimetics (e.g., epinephrine and dipivalylepinephrine); beta-blockers (e.g., betaxolol, levobunolol and timolol); alpha-2 agonists (e.g., para-amino clonidine); carbonic anhydrase inhibitors (e.g., acetazolamide, methazolamide and ethoxzolamide); and prostaglandins and their analogs and derivatives (e.g., latanaprost) in a single formulation for administration. One skilled in the art will recognize due care will need to be given in selecting such agents for co-administration from a single formulation with due regard for chemical stability and compatibility with other agents (whether active therapeutic agents or excipients) in the composition made available to the patient.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following example is provided by way of illustration only and not by way of limitation. Those of skill in the art will readily recognize a variety of noncritical parameters that could be changed or modified to yield essentially similar results.

### Example 1

### Efficacy Study in Normotensive Rabbits—Topical Administration

The time-related changes in IOP in response to a topically administered formula V suspensions or vehicle were determined in pigmented rabbits. The vehicle formulation consisted of (w/v): 0.06% carbomer (carbopol 1382) NF, 1.0% poloxamer 188, NF, 2.5% glycerin, USP, 0.01% benzalkonium chloride, NF, sodium hydroxide, NF (q.s. pH 7.4), and purified water, USP (q.s. 100%).

The suspensions consisted of (w/v): 1% compound of formula V, 0.06% carbomer (carbopol 1382) NF, 1.0% poloxamer 188, NF, 2.5% glycerin, USP, 0.01% benzalkonium chloride, NF, sodium hydroxide, NF (q.s. pH 7.4), and purified water, USP (q.s. 100%) or 0.2% compound of formula V, 0.06% carbomer (carbopol 1382) NF, 1.0% poloxamer 188, NF, 2.5% glycerin, USP, 0.01% benzalkonium chloride, NF, sodium hydroxide, NF (q.s. pH 7.4), and purified water, USP (q.s. 100%).

IOPs (mmHg) were measured using a monometrically calibrated Digilab Model 30-D pneumatonometer (Biorad). Pupil diameters (mm) were measured horizontally utilizing an Opstick (Allergan, Irvine, Calif.). Tetracaine 0.5% (25 μL) was applied to each eye prior to IOP measurements. Two baseline readings were taken at −0.5 and 0 hours before vehicle or test article administration. Subsequently, measurements were done at 0.5, 1, 2, 3, 4, 5, and 6 hours post-test article. At the end of the day's measurements, stability of the tonometer was confirmed using the verifier supplied by Mentor.

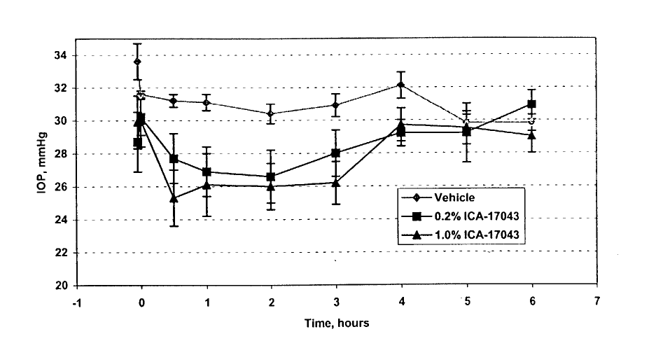

As shown in FIG. 1, there was a statistically significant decrease in IOP observed after the administration of 0.2% suspension beginning at 30 min and lasting until the 4 hour measurement. The maximum change was between the time 0 measurement and the 2 hour measurement, 3.4 mmHg. There was also a statistically significant decrease in IOP following administration of the 1.0% suspension. Again, IOP was decreased between 30 min and 4 hours, with a maximum decrease of 4.7 mmHg observed 30 minutes post administration compared to the time 0 measurement. Compared to vehicle effects in the same animals, IOP values were as much as 6 mmHg lower in formula V treated animals at a given time point.

What is claimed is:

1. A method for reducing intraocular pressure in a subject in need thereof by decreasing potassium ion flow through intermediate conductance potassium (IK1) channels in a cell, the method comprising the step of administering to the subject a pharmaceutically acceptable carrier and a compound able to decrease potassium ion flow through IK1 channels, said composition administered to the subject in a potassium ion flow decreasing amount, thereby reducing intraocular pressure.

2. The method of claim 1, wherein the subject has glaucoma characterized by increased intraocular pressure.

3. The method of claim 1, wherein the method prevents glaucoma characterized by increased intraocular pressure.

4. The method of claim 2, wherein the glaucoma is primary open-angle glaucoma, normal tension glaucoma, angle-closure glaucoma, acute glaucoma, pigmentary glaucoma, neovascular glaucoma, or trauma related glaucoma.

5. The method of claim 1, wherein the subject has increased intraocular pressure associated with Sturge-Weber syndrome.

6. The method of claim 1, wherein the method prevents increased intraocular pressure associated with Sturge-Weber syndrome.

7. The method of claim 1, wherein the subject has increased intraocular pressure caused by uveitis.

8. The method of claim 1, wherein the method reduces intraocular pressure to between about 12 and 20 mm of mercury.

9. The method of claim 1, wherein the method maintains intraocular pressure between about 12 and 20 mm of mercury.

10. The method of claim 1, wherein the compound treats chronic elevation of intraocular pressure.

11. The method of claim 1, wherein the compound treats acute elevation of intraocular pressure.

12. The method of claim 1, wherein the compound treats gradual elevation of intraocular pressure.

13. The method of claim 1, wherein the method prevents destruction of optic nerve cells.

14. The method of claim 1, wherein the method prevents atrophy of optic nerve cells.

15. The method of claim 1, wherein the method prevents blindness.

16. The method of claim 1, wherein the compound treats exfoliation syndrome characterized by increased intraocular pressure.

17. The method of claim 1, wherein the subject is a human.

18. The method of claim 1, wherein the potassium ion flow decreasing amount is about 0.001% to 10% w/v.

19. The method of claim 1, wherein the potassium ion flow decreasing amount is about 0.1% to 5% w/v.

20. The method of claim 1, wherein the potassium ion flow decreasing amount is about 10-1000 μg per eye.

21. The method of claim 1, wherein the potassium ion flow decreasing amount is about 75-150 μg per eye.

22. The method of claim 1, wherein the composition is administered topically.

23. The method of claim 1, further comprising the step of administering to the subject a second pharmaceutical composition known to reduce intraocular pressure in a subject.

24. The method of claim 23, wherein the second pharmaceutical composition comprises as its active ingredient one or more compounds selected from the group consisting of timolol, betaxolol, levobunolol, acetazolamide, methazolamide, dichlorphenamide, dorzolamide, brinzolamide, latanoprost, brimonidine, or bimatoprost.

25. The method of claim 1, wherein the compound able to decrease ion flow through IK1 channels has the formula: 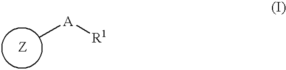
wherein
ring system Z is a member selected from the group consisting of substituted or unsubstituted aryl, and substituted or unsubstituted 5-membered heterocycle;
A is a member selected from —NHS(O)₂—, —S(O)₂NH—, —C(R³R⁴)S(O)ₙ, and —S(O)ₙC(R³R⁴)—, wherein R³ and R⁴ are members independently selected from the group consisting of hydrogen, substituted or unsubstituted lower alkyl, OR⁵ and —CF₃, wherein R⁵ is a member selected from hydrogen, substituted or unsubstituted lower alkyl, and CF₃;
n is selected from the integers from 0 to 2; and
R¹ is a member selected from the group of substituted or unsubstituted aryl, substituted or unsubstituted heteroaryl, substituted or unsubstituted (C₅-C₇)carbocycle, and substituted or unsubstituted (C₅-C₇)heterocycle.

26. The method of claim 25, wherein A is —NHS(O)₂—.

27. The method of claim 25, wherein the compound able to decrease ion flow through IK1 channels has the formula: 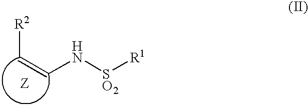
wherein
ring system Z is a member selected from the group consisting of substituted or unsubstituted aryl, and substituted or unsubstituted 5-membered heterocycle;
R¹ is a member selected from the group of substituted or unsubstituted aryl, substituted or unsubstituted heteroaryl, substituted or unsubstituted (C₅-C₇)carbocycle, and substituted or unsubstituted (C₅-C₇)heterocycle;
R² is a member selected from COOR⁶, substituted or unsubstituted 2-furan, substituted or unsubstituted 2-thiazole, and 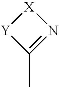
wherein
R⁶ is a member selected from the group consisting of methyl, ethyl, and —CF₃;
X is selected from the group consisting of —N═N—, —N═C(R⁷)—, —C(R⁷R⁸)—C(R⁷R⁸)—, and —C(R⁷)═C(R⁸)—, wherein R⁷ and R⁸ are members independently selected from the group consisting of hydrogen, substituted and unsubstituted lower alkyl, and —CF₃; and
Y is a member selected from O, —NR⁹—, and S, wherein R⁹ is a member selected from —H, lower alkyl, and —CF₃.

28. The method of claim 25, wherein the compound able to decrease ion flow through IK1 channels is selected from the group consisting of:

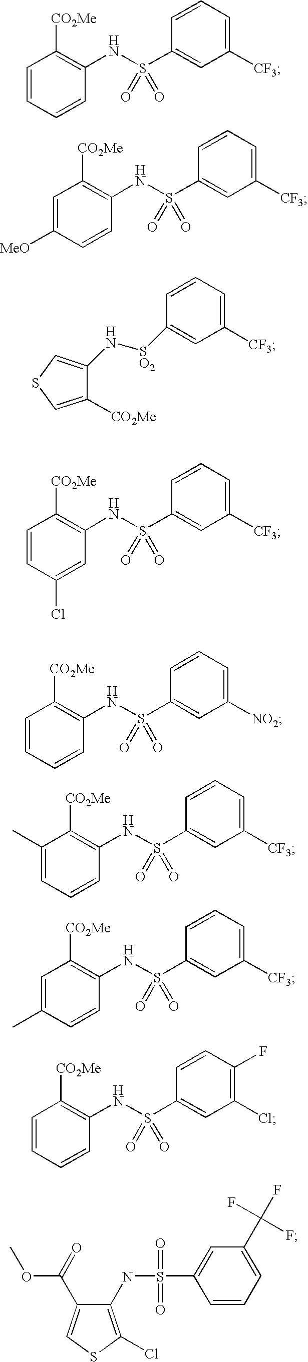

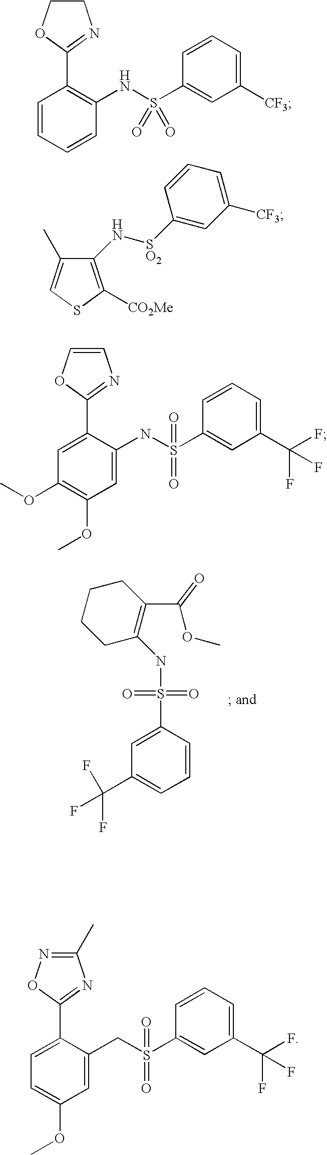

29. The method of claim 25, further comprising the step of administering to the subject a second pharmaceutical composition known to reduce intraocular pressure in a subject.

30. The method of claim 29, wherein the second pharmaceutical composition comprises as its active ingredient one or more compounds selected from the group consisting of timolol, betaxolol, levobunolol, acetazolamide, methazolamide, dichlorphenamide, dorzolamide, brinzolamide, latanoprost, brimonidine, or bimatoprost.

31. The method of claim 1, wherein the compound able to decrease ion flow through IK1 channels has the formula: 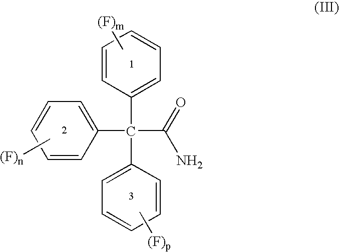
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

32. The method of claim 31, wherein the compound able to decrease ion flow through IK1 channels has the formula: 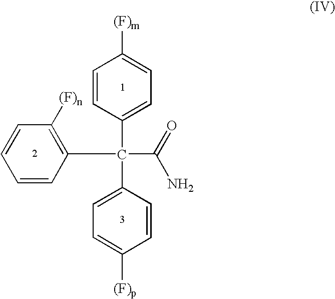
wherein,
m, n and p are independently selected from 0 and 1, and at least one of m, n and p is 1.

33. The method of claim 32, wherein the compound able to decrease ion flow through IK1 channels has the formula: 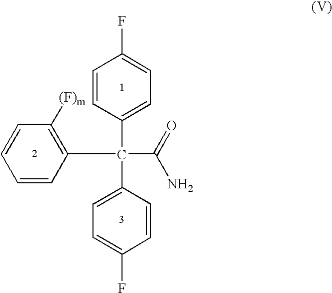
wherein m is either 0 or 1.

34. The method of claim 33, wherein the compound able to decrease ion flow through IK1 channels is selected from the group consisting of:

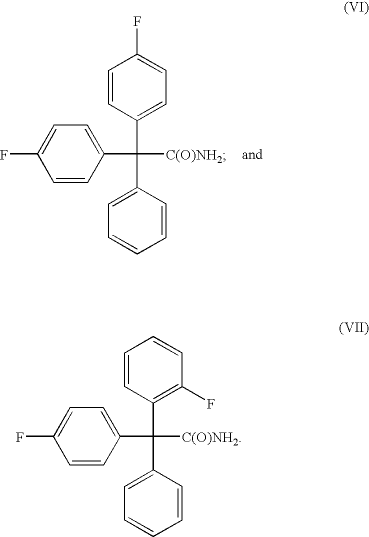

35. The method of claim 34, wherein the compound able to decrease ion flow through IK1 channels has the formula (VIII):

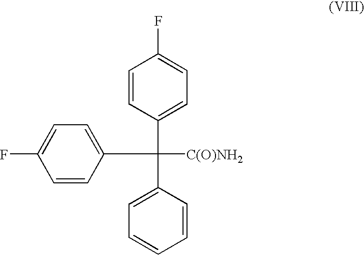

36. The method of claim 35, further comprising the step of administering to the subject a second pharmaceutical composition known to reduce intraocular pressure in a subject.

37. The method of claim 36, wherein the second pharmaceutical composition comprises as its active ingredient one or more compounds selected from the group consisting of timolol, betaxolol, levobunolol, acetazolamide, methazolamide, dichlorphenamide, dorzolamide, brinzolamide, latanoprost, brimonidine, or bimatoprost.

38. The method of claim 31, further comprising the step of administering to the subject a second pharmaceutical composition known to reduce intraocular pressure in a subject.

39. The method of claim 38, wherein the second pharmaceutical composition comprises as its active ingredient one or more compounds selected from the group consisting of timolol, betaxolol, levobunolol, acetazolamide, methazolamide, dichlorphenamide, dorzolamide, brinzolamide, latanoprost, brimonidine, or bimatoprost.

40. The method of claim 1, wherein the compound is administered as a pharmaceutical composition comprising the following elements in a pharmaceutically acceptable carrier: 1.0% of the compound, micronized; 0.06% carbomer; 1.0% poloxamer 188; 2.5% glycerin; 0.01% benzalkonium chloride; sodium hydroxide, q.s. pH 7.4±0.3; and purified water.

41. The method of claim 34, wherein the compound of formula V is administered as a pharmaceutical composition comprising the following elements in a pharmaceutically acceptable carrier: 1.0% of the compound, micronized; 0.06% carbomer; 1.0% poloxamer 188; 2.5% glycerin; 0.01% benzalkonium chloride; sodium hydroxide, q.s. pH 7.4±0.3; and purified water.

42. A pharmaceutical formulation comprising a pharmaceutically acceptable excipient and a compound having the formula: 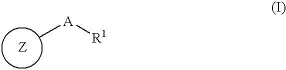
wherein
ring system Z is a member selected from the group consisting of substituted or unsubstituted aryl, and substituted or unsubstituted 5-membered heterocycle;
A is a member selected from —NHS(O)₂—, —S(O)₂NH—, —C(R³R⁴)S(O)ₙ, and —S(O)ₙC(R³R⁴)—, wherein R³ and R⁴ are members independently selected from the group consisting of hydrogen, substituted or unsubstituted lower alkyl, OR⁵ and —CF₃, wherein R⁵ is a member selected from hydrogen, substituted or unsubstituted lower alkyl, and CF₃;
n is selected from the integers from 0 to 2; and
R¹ is a member selected from the group of substituted or unsubstituted aryl, substituted or unsubstituted heteroaryl, substituted or unsubstituted (C₅-C₇)carbocycle, and substituted or unsubstituted (C₅-C₇)heterocycle.

43. The formulation of claim 42, wherein the compound has formula I in which symbol A has the formula —NHS(O)₂—.

44. The formulation of claim 42, wherein the compound has the formula: 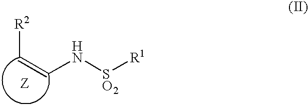
wherein
ring system Z is a member selected from the group consisting of substituted or unsubstituted aryl, and substituted or unsubstituted 5-membered heterocycle;
R¹ is a member selected from the group of substituted or unsubstituted aryl, substituted or unsubstituted heteroaryl, substituted or unsubstituted (C₅-C₇)carbocycle, and substituted or unsubstituted (C₅-C₇)heterocycle;
R² is a member selected from COOR⁶, substituted or unsubstituted 2-furan, substituted or unsubstituted 2-thiazole, and 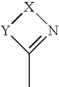
wherein
R⁶ is a member selected from the group consisting of methyl, ethyl, and —CF₃;
X is selected from the group consisting of —N═N—, —N═C(R⁷)—, —C(R⁷R⁸)—C(R⁷R⁸)—, and —C(R⁷)═C(R⁸)—, wherein R⁷ and R⁸ are members independently selected from the group consisting of hydrogen, substituted and unsubstituted lower alkyl, and —CF₃; and
Y is a member selected from O, —NR⁹—, and S, wherein R⁹ is a member selected from —H, lower alkyl, and —CF₃

45. The formulation of claim 42, further comprising a second compound known to reduce intraocular pressure in a subject.

46. The formulation of claim 45, wherein the second compound is selected from the group consisting of timolol, betaxolol, levobunolol, acetazolamide, methazolamide, dichlorphenamide, dorzolamide, brinzolamide, latanoprost, brimonidine, or bimatoprost.

47. A pharmaceutical formulation comprising a pharmaceutically acceptable excipient and a compound having the formula: 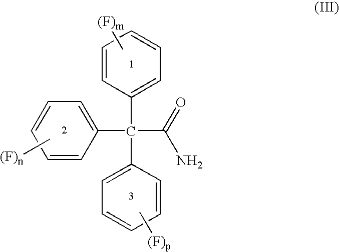
wherein,
m, n and p are independently selected from 0 and 1 and at least one of m, n and p is 1;
when m, n and p are all 1, the fluoro substituents at ring 1 and at ring 2 are located at a position independently selected from ortho to the acetamide substituent, meta to the acetamide substituent and para to the acetamide substituent, and the substituent at ring 3 is at a position selected from ortho to the acetamide substituent and para to the acetamide substituent; and
when p is 0, and m is 1 and n is 1, the fluoro substituent at ring 1 is para to the acetamide substituent, and the substituent at ring 2 is located at a position selected from ortho to the acetamide substituent and para to the acetamide substituent.

48. The formulation of claim 47, further comprising a second compound known to reduce intraocular pressure in a subject.

49. The formulation of claim 48, wherein the second compound is selected from the group consisting of timolol, betaxolol, levobunolol, acetazolamide, methazolamide, dichlorphenamide, dorzolamide, brinzolamide, latanoprost, brimonidine, or bimatoprost.
